(19) United States
(12) Patent Application Publication
(10) Pub. No.: US 2025/0000686 A1
(43) Pub. Date: Jan. 2, 2025

(54) FLUID COLLECTION ASSEMBLIES INCLUDING AT LEAST ONE INFLATION DEVICE AND METHODS AND SYSTEMS OF USING THE SAME

(71) Applicant: PUREWICK CORPORATION, Covington, GA (US)

(72) Inventors: Jason Jishen Cheng, Rochester, NY (US); Eric Rehm, Greensboro, GA (US); Nicholas Austerman, Atlanta, GA (US)

(21) Appl. No.: 18/758,025

(22) Filed: Jun. 28, 2024

Related U.S. Application Data
(63) Continuation of application No. 17/326,980, filed on May 21, 2021, now Pat. No. 12,048,643.
(60) Provisional application No. 63/030,685, filed on May 27, 2020.

(51) Int. Cl. A61F 5/451; A61F 5/44; A61F 5/453; A61F 5/455; A61M 1/00

(52) U.S. Cl. CPC A61F 5/451; A61F 5/4405; A61F 5/453; A61F 5/455; A61M 1/80

(57) ABSTRACT

Embodiments disclosed herein are directed to fluid collection assemblies that include at least one inflation device, methods for using the same, and systems including the same. An example fluid collection assembly includes a fluid impermeable barrier defining at least one opening, a chamber, and at least one fluid outlet. The fluid collection assembly also includes at least one porous material disposed in the chamber. The fluid collection assembly further includes at least one inflation device. The inflation device includes at least one bladder having one or more walls defining an interior region. The inflation device also includes at least one valve configured to selectively permit flow of at least one inflation fluid into and out of the interior region to switch the at least one bladder between a first state and a second state.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden patients are sometimes used. However, bedpans can be prone to discomfort, spills, and other hygiene issues.

### SUMMARY

Embodiments disclosed herein are directed to fluid collection assemblies that include at least one inflation device, methods for using the same, and systems including the same. In an embodiment, a fluid collection assembly is disclosed. The fluid collection assembly includes a fluid impermeable barrier defining a chamber, at least one opening, and at least one fluid outlet. The fluid collection assembly also includes at least one porous material disposed in the chamber. The fluid collection assembly further includes at least one inflation device including at least one bladder and at least one valve. The at least one bladder includes one or more walls defining an interior region. The at least one valve is configured to selectively permit at least one inflation fluid to flow into and out of the interior region to switch the at least one bladder between a first state and a second state. An amount of the at least one inflation fluid present in the interior region is greater when the at least one bladder is in the second state than when the at least one bladder is in the first state.

In an embodiment, a system is disclosed. The system includes a fluid collection assembly, a fluid storage container, a vacuum source, and one or more conduits. The one or more conduits operably couple at least one fluid outlet of the fluid collection assembly, the fluid storage container, and the vacuum source together. The fluid collection assembly of the system includes any one of the fluid collection assemblies disclosed herein.

In an embodiment, a method of using a fluid collection assembly is disclosed. The method includes positioning at least one opening of the fluid collection assembly adjacent to a female urethra or to receive a male penis therethrough. The fluid collection assembly includes a fluid impermeable barrier defining the at least one opening, a chamber, and the at least one fluid outlet. The fluid collection assembly also includes at least one porous material disposed in the chamber. The fluid collection assembly further includes at least one inflation device including at least one bladder and at least one valve, the at least one bladder including one or more walls defining an interior region. The method includes flowing at least one inflation fluid through the at least one valve and into the interior region of the at least one inflation element.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly configured to be used to collect bodily fluids from a female urethral opening, according to an embodiment.

FIGS. 1B and 1C are schematic cross-sectional views of the fluid collection assembly taken along plane 1B-1B when a bladder of the fluid collection assembly is in a first state and in a second state, respectively, according to an embodiment.

FIG. 1D is a schematic cross-sectional view of the fluid collection assembly taken along plane 1D-1D, according to an embodiment.

FIGS. 2A to 2C are schematic cross-sectional views of different fluid collection assemblies that each have inflation devices positioned differently, according to different embodiments.

FIG. 3 is a cross-sectional schematic view of a fluid collection assembly when the at least one bladder thereof is in the second state, according to an embodiment.

FIGS. 4A to 4C are schematic cross-sectional views of fluid collection assemblies exhibiting different shapes when the bladders of the fluid collection assemblies are in the second state, according to different embodiments.

FIG. 5A is a schematic illustration of a first fluid collection assembly being used with a first female patient, according to an embodiment.

FIG. 5B is a schematic illustrated of a second fluid collection assembly being used with a second female patient, according to an embodiment.

FIG. 6 is a cross-sectional view of a male fluid collection assembly, according to an embodiment.

FIG. 7 is a block diagram of a system for fluid collection, according to an embodiment.

### DETAILED DESCRIPTION

Embodiments disclosed herein are directed to fluid collection assemblies that include at least one inflation device, methods for using the same, and systems including the same. An example fluid collection assembly includes a fluid impermeable barrier defining at least one opening, a chamber, and at least one fluid outlet. The fluid collection assembly also includes at least one porous material disposed in the chamber. As previously discussed, the fluid collection assembly further includes at least one inflation device. The inflation device includes at least one bladder having one or more walls defining an interior region. The inflation device also includes at least one valve configured to selectively permit fluid flow into and out of the interior region to switch the at least one bladder between a first state (e.g., a deflated and/or initial state) and a second state (e.g., an at least partially inflated state). The inflation device may facilitate securement of the fluid collection assembly with certain patients (e.g., underweight patients or patients with thin thighs) when the bladder of the inflation device is in the second state.

The fluid collection assembly is configured to be positioned about or around the pubic region of a patient. When the patient is a female, the pubic region may include the vulva (e.g., labia majora, labia minora, clitoris, urethral opening, etc.), mons pubis, the perineum, buttocks, and the inner thighs of the patient. When the patient is a male, the pubic region may include the penis, the testicles, the mons pubis, the buttocks, and the inner thighs of the patient.

The fluid collection assembly may be flexible thereby allowing the fluid collection assembly to conform to the size and topography of the pubic region since the size and topography of the pubic region may vary significantly from person to person. Conforming the fluid collection assembly to the pubic region may prevent or inhibit embarrassing and unsanitary leaks of bodily fluids (e.g., urine, blood, sweat, etc.). In some embodiments, the fluid collection assembly may rely on contact pressure from the thighs of the individual to remain conformed to the pubic region. However, it has been found that the thighs of some individuals may not provide sufficient contact pressure to allow the fluid collection assembly to remain conformed to the pubic when the fluid collection assembly is in the first (e.g., deflated) state. For instance, it has been found that underweight patients and other patients having thin thighs may not provide sufficient contact pressure to the fluid collection assembly to allow the fluid collection assembly to remain conformed to the pubic region when the fluid collection assembly is in the first state. Some conventional fluid collection assemblies that do not include the inflation device may have similar problems remaining conformed to the pubic region.

The inflatable devices disclosed herein allow the fluid collection assemblies disclosed herein to remain conformed to the pubic region even when the patient is underweight or has thin thighs. For example, when the fluid collection assembly does not have sufficient contact pressure from the thighs to remain conformed to the pubic region, at least one inflation fluid (e.g., gas or liquid) may be added to the interior region of the bladder through the valve. Adding the fluid to the interior region switches the bladder from the first state to the second state. Changing the bladder from the first state to the second shape also changes the shape and/or size of the fluid collection assembly. The changes in the shape and/or size of the fluid collection assembly may allow the fluid collection assembly to press against the thighs so the thighs provide sufficient contact pressure to allow the fluid collection assembly to remain conformed to the female anatomy.

The fluid collection assembly is configured to be disposed against the patient so the opening of the fluid impermeable barrier is disposed adjacent to at least the urethral opening of the patient. After positioning the fluid collection assembly, the patient may discharge bodily fluids, either controllably or uncontrollably. The porous material may remove the bodily fluids from the patient and move the bodily fluids through the opening and the chamber to the fluid outlet. The bodily fluids may be removed from the chamber via the fluid outlet by using at least one gravity (e.g., the fluid outlet is at the gravimetric low point of the chamber) or a suction force provided by a vacuum which suctions the bodily fluids from the chamber.

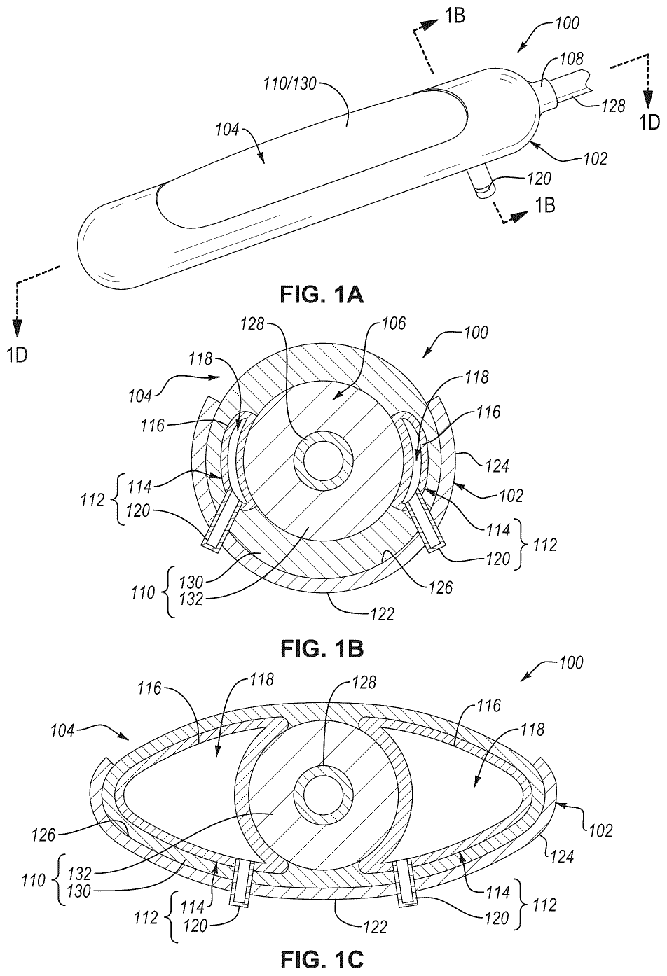

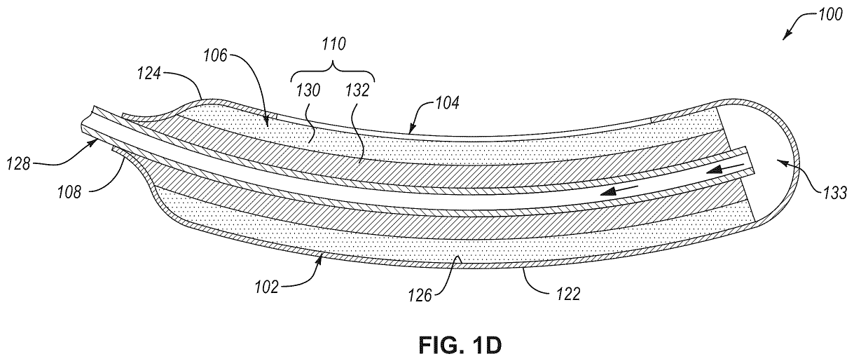

FIG. 1A is an isometric view of a fluid collection assembly 100 configured to be used to collect bodily fluids from a female urethral opening, according to an embodiment. FIGS. 1B and 1C are schematic cross-sectional views of the fluid collection assembly 100 taken along plane 1B-1B when a bladder 114 of the fluid collection assembly 100 is in a first state and in a second state, respectively, according to an embodiment. FIG. 1D is a schematic cross-sectional view of the fluid collection assembly 100 taken along plane 1D-1D, according to an embodiment. The fluid collection assembly 100 includes a fluid impermeable barrier 102 defining at least one opening 104, a chamber 106, and at least one fluid outlet 108. The fluid collection assembly 100 also includes at least one porous material 110 disposed chamber 106. The fluid collection assembly 100 further includes at least one inflation device 112 configured to change the shape and/or size of the fluid collection assembly 100.

The inflation device 112 includes a bladder 114. The bladder 114 includes one or more walls 116 defining an interior region 118. The inflation device 112 also includes at least one valve 120 in fluid communication with the interior region 118. The valve 120 is configured to selectively permit flow of an inflation fluid into and/or out the interior region 118. For example, the valve 120 may allow an inflation fluid to enter the interior region 118 when it is desirable to at least one of increase the size or change the shape of the bladder 114 which, in turn, at least one of increases the size or changes the shape of the fluid collection assembly 100. The valve 120 may also enable removing the inflation fluid from the interior region 118 when it is desirable to at least one of decrease the size or change the shape (e.g., return to the initial shape) of the bladder 114 which, in turn, at least one of decreases the size or changes the shape of the fluid collection assembly 100.

Disposing or removing the inflation fluids into and from the inflation region 118 changes the state of the bladder 114. The bladder 114 may exhibit at least a first state and a second state. The amount (volume or weight) of inflation fluids present in the inflation region 118 is greater when the bladder 114 is in the second state than when the bladder 114 is in the first state. In an example, as shown in FIG. 1B, the bladder 114 is in the first state when the bladder 114 is in a deflated state (e.g., there are no or substantially no fluids in the inflation region 118). However, it is noted that the bladder 114 may be in the first state when some inflation fluids are present in the inflation region 118. The bladder 114 generally also correspond to an initial state of the bladder 114 (i.e., generally, the bladder 114 is not provided with inflation fluids) though, in some examples, the first state of the bladder 114 may not correspond to the initial state of the bladder 114 (e.g., the bladder 114 is provided with fluids). In an example, as shown in FIG. 1C, the bladder 114 is in the first state when the bladder 114 is in an at least partially inflated state. The bladder 114 is generally not in the initial state when the bladder 114 is in the second state though, in some examples, the initial state and the second state of the bladder 114 are the same (e.g., when the bladder 114 is provided with fluids).

The bladder 114 may exhibit one or more additional states (e.g., third state, fourth state, and so forth) besides the first and second states discussed above. In an embodiment, the one or more additional states may include less inflation fluids in the inflation region 118 (e.g., is more deflated) than the first state (e.g., the first state is a partially inflated state). In such an embodiment, the one or more additional states may include a deflated or partially deflated state and may be formed by removing inflation fluids from the inflation region 118 when the bladder 114 is in the first or second state. In an embodiment, the one or more additional states may include more inflation fluids in the inflation region 118 (e.g., is more inflated) than the first state (e.g., the first state is a deflated or partially inflated state) and include less inflation fluids in the inflation region 118 than the second state. In such an embodiment, the one or more additional states include a partially inflated state and may be formed by adding or removing inflation fluids to the inflation region 118 when the bladder 114 is in the first state or second state, respectively. In an embodiment, the one or more additional states may include more inflation fluids in the inflation region 118 than the second state (e.g., the second state is a partially inflated state). In such an embodiment, the one or more additional states may be an at least partially inflated state and may be formed by adding inflation fluids to the inflation region 118 when the bladder 114 is in the first state or second state. It is noted that, in some embodiments, the bladder 114 may only include the first and second states.

As shown in FIGS. 1B and 1C, switching the bladder 114 from the first state to the second state (or any of the other states thereof) changes the shape and/or size of the fluid collection assembly 100 and the bladder 114. For example, referring to FIG. 1B, the fluid collection assembly 100 may exhibit a generally circular cross-sectional shape (e.g., the fluid collection assembly 100 exhibits a generally cylindrical shape, as shown in FIG. 1A) when the bladder 114 is in the first state. The bladder 114 may also exhibit a thin generally crescent cross-sectional shape when the bladder 114 is in the first state. It is noted that the fluid collection assembly 100 and/or the bladder 114 may exhibit different shapes when the bladder 114 is in the first state than the shapes illustrated in FIG. 1B. Switching the bladder 114 to the second state changes the shape of the fluid collection assembly 100, as shown in FIG. 1C, to a generally oblong cross-sectional shape and the bladder 114 into a wide generally crescent shape (i.e., a generally triangular shape). Further, switching the bladder 114 from the first state to the second state increases the size of the fluid collection assembly 100 and the bladder 114. For example, the increased size of the fluid collection assembly 100 may allow the fluid collection assembly 100 to contact and press against the inner thighs of a patient, even an underweight patient or a patient with thin thighs, allowing the fluid collection assembly 100 to remain conformed to the pubic region of the patient. It is noted that inflation fluids may be removed from the inflation region 118 using the valve 120, for example, when too much inflation fluids are added to the inflation region 118 which may at least one of increase the risk that the bladder 114 ruptures, make the fluid collection assembly 100 more uncomfortable, or increase the likelihood that the fluid collection assembly 100 leaks bodily fluids.

The walls 116 are formed from a material is substantially impermeable to the inflation fluid (e.g., substantially impermeable to a gas and/or a liquid) which allows the bladder 114 to retain the inflation fluids without embarrassing leaks. The walls 116 may also be formed from a flexible material. The flexible material of the walls 116 allows the bladder 114 and, by extension, the fluid collection assembly 100 to at least one of increase in size or change a shape thereof. For example, the flexible material of the walls 116 allow the interior region 118 to increase a volume thereof when the interior region 118 receives an inflation fluid and decrease a volume thereof when inflation fluids are removed from the interior region 118. Examples of materials that may form the walls 116 of the bladder 114 include silicone, rubber, latex, polychloroprene, nylon fabric, polypropylene, polyvinyl chloride, nitrile rubber, other suitable polymers, a metal foil, a composite, or combinations thereof. It is noted that the walls 116 do not contact the patient in the illustrated embodiment and might be formed from a biocompatible material. In an embodiment, the walls 116 are configured to stretch (e.g., elastically or plastically stretch) so the walls 116 remain taut when the bladder 114 is at least partially inflated. In an embodiment, the wall 116 forms a plurality of wrinkles when the bladder 114 is at least partially deflated and adding inflation fluid into the inflation region 118 decreases the wrinkles, similar to an accordion.

The valve 120 may include any suitable valve configured to allow for the controllable addition and remove of inflation fluids from the inflation region 118. In an embodiment, the valve 120 is a luer valve and includes a male-tapper fitting or a female-taper fitting. In an embodiment, the valve 120 includes a fluid impermeable membrane with a slit or opening formed. The slit or opening of the fluid impermeable membrane remains substantially closed when no external load is applied thereto but opens when an external load is applied thereto (e.g., an external load caused by pressing a syringe against the fluid impermeable membrane). In an embodiment, the valve 120 may include a mechanical valve, such as a ball valve, a butterfly valve, or any other suitable mechanical valve. The mechanical valve may be manually operated or controlled using a computer. In an embodiment, the valve 120 may include a check valve to limit leaks from the bladder 114 and to make the fluid collection assembly 100 easier to use. In such an embodiment, the valve 120 may only add or remove (but not both) inflation fluid from the inflation region 118 and, as such, the fluid collection assembly 100 is configured for single use.

In an embodiment, as illustrated, the valve 120 may extend outwardly from the bladder 114. For example, the valve 120 may extend from the bladder 114, through a portion of the porous material 110, and through the fluid impermeable barrier 102 thereby allowing a user (e.g., medical practitioner or patient) of the fluid collection assembly 100 easily access to the valve 120. As shown, the valve 120 may extend a short distance only from the fluid impermeable barrier 102, such as about 2 cm or less, about 1.5 cm or less, about 1 cm or less, about 0.75 cm or less, about 0.5 cm or less, about 0.25 cm or less, or in ranges of about 0.25 cm to about 0.75 cm, about 0.5 cm to about 1 cm, about 0.75 cm to about 1.5 cm, or about 1 cm to about 2 cm. However, the valve 120 may extend a significant distance from the fluid impermeable barrier 102, such as a distance that is about 2 cm or greater, about 5 cm or greater, about 10 cm or greater, about 50 cm or greater, about 100 cm or greater, about 500 cm or greater, about 1 m or greater, about 2 m or greater, or in ranges of about 2 cm to about 10 cm, about 5 cm to about 50 cm, about 10 cm to about 100 cm, about 50 cm to about 500 cm, about 100 cm to about 1 m, or about 500 cm to about 2 m. When the valve 120 extends a significant distance from the fluid impermeable barrier 102, the valve 120 may include a flexible tube which allows a user of the fluid collection assembly 100 to easily access to the valve 120 while the fluid collection assembly 100 is positioned adjacent to the pubic region without having the user near the pubic region (which the patient may find uncomfortable).

In an embodiment, as illustrated, the valve 120 extends from or near a back surface 122 of the fluid impermeable barrier 102. The back surface 122 of the fluid impermeable barrier 102 is the surface of the fluid impermeable barrier 102 opposite the opening 104. The valve 120 at or near the back surface 122 may allow a user of the fluid collection assembly 100 to access the valve 120 when the fluid collection assembly 100 is adjacent to the pubic region since, generally, the pubic region and the inner thighs of the patient may contact or obstruct the surfaces of the fluid impermeable barrier 102 except the back surface 122. Further, the valve 120 at or near the back surface 122 prevents the valve 120 from pressing against the pubic region and the inner thighs during use which may cause discomfort.

In an embodiment, the fluid collection assembly 100 may only include a single bladder 114 and/or a single valve 120. In an embodiment, the fluid collection assembly 100 may include a plurality of bladders 114 and/or a plurality of valves 120. For example, as illustrated, the fluid collection assembly 100 may include two bladders 114 on each lateral side of the fluid collection assembly 100 which allows the fluid collection assembly 100 to increase is size laterally which allows the fluid collection assembly 100 to contact the thighs of the patient. Each bladder 114 may include one or more valves 120 to allow for independent inflation of the bladders 114 which allows for better control of the shape and size of the fluid collection assembly 100. However, the fluid collection assembly 100 may only include a single valve 120 for two or more bladders 114. In some examples, a single bladder 114 may include a plurality of valves 120, for instance, to increase the likelihood that one valve 120 is easily accessible.

The inflation device 112 may include additional components other than the components discussed above. For example, the inflation device 112 may include less flexible material (e.g., rigid material or material that is less flexible than the walls 116) that are used to control the shape of the bladder 114 when the bladder 114 is inflated (e.g., switched from the first state to the second state). The less flexible material may form parts of the walls 116, wrap around the walls 116, or extend in the inflation region 118 between opposing portions of the walls 116 which limits expansion of the walls 116 at and near the less flexible material.

The at least one inflation fluid added or removed from the interior region 118 may include any suitable fluid, such as any suitable liquid or any suitable gas. In an embodiment, the inflation fluids are formed from a generally regarded as safe (“GRAS”) material. Forming the inflation fluids from a GRAS materials may decrease health risks caused by inadvertently exposing the patient to the inflation fluids. Examples of GRAS materials that may form the inflation fluids includes water, saline solution, alcohol solution, atmospheric air, nitrogen, any other GRAS material, or combinations thereof.

As previously discussed, the fluid collection assembly 100 includes a fluid impermeable barrier 102. In the illustrated embodiment, the fluid impermeable barrier 102 is distinct from the inflation device 112 and is spaced from the inflation device 112 by at least a portion of the porous material 110.

The fluid impermeable barrier 102 may be formed of any suitable fluid imperious material(s), such as a fluid impermeable polymer (e.g., silicone, polypropylene, polyethylene, polyethylene terephthalate, a polycarbonate, etc.), a metal film, natural rubber, another suitable material, or combinations thereof. The fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least a surface of the fluid impermeable barrier 102 that may contact the patient may be formed from a soft and/or smooth material (e.g., silicone), thereby reducing chaffing. In an embodiment, the fluid impermeable barrier 102 may be formed from a flexible material, such as silicone, which allows the fluid impermeable barrier 102 to be bent into a shape that conforms the anatomy of the individual. Further, as shown in FIGS. 1B and 1C, forming the fluid impermeable barrier 102 from a flexible material allows the fluid impermeable barrier 102 to accommodate the shape and/or size changes by switching the fluid collection assembly 100 and the bladder 114 between states.

The fluid impermeable barrier 102 may define a hole that allows the valve 120 to extend through the fluid impermeable barrier 102. For example, the fluid impermeable barrier 102 may define the hole at or near the back surface 122 thereof. The hole formed in the fluid impermeable barrier 102 is configured to form a substantially fluid tight seal against the valve 120 or a sealant is applied between the fluid impermeable barrier 102 and the valve 120 to prevent bodily fluids from leaking through the hole.

In some examples, the fluid impermeable barrier 102 may be tubular (ignoring the opening), such as substantially cylindrical (as shown), oblong, prismatic, or flattened tubes when the bladder 114 is in the first state and/or the second state. During use, the outer surface 124 of the fluid impermeable barrier 102 may contact the patient. The fluid impermeable barrier 102 may be sized and shaped to fit in the gluteal cleft between the legs of a female user when the bladder 114 are in at least the second state.

The opening 104 provides an ingress route for fluids to enter the chamber 106. The opening 104 may be defined by the fluid impermeable barrier 102 such as by an inner edge of the fluid impermeable barrier 102. For example, the opening 104 is formed in and extends through the fluid impermeable barrier 102, from the outer surface 124 to the inner surface 126, thereby enabling fluid(s) to enter the chamber 106 from outside of the fluid collection assembly 100. The opening 104 may be an elongated hole in the fluid impermeable barrier 102. For example, the opening 104 may be defined as a cut-out in the fluid impermeable barrier 102. The opening 104 may be located and shaped to be positioned adjacent to a female urethra.

The fluid collection assembly 100 may be positioned proximate to the female urethral opening and urine may enter the chamber of the fluid collection assembly 100 via the opening 104. The fluid collection assembly 100 is configured to receive the fluid(s) into the chamber 106 via the opening 104. When in use, the opening 104 may have an elongated shape that extends from a first location below the urethral opening (e.g., at or near the anus or the vaginal opening) to a second location above the urethral opening (e.g., at or near the top of the vaginal opening or the pubic hair).

The opening 104 may have an elongated shape because the space between the legs of a female is relatively small when the legs of the female are closed, thereby only permitting the flow of the fluid(s) along a path that corresponds to the elongated shape of the opening 104 (e.g., longitudinally extending opening). The opening 104 in the fluid impermeable barrier 102 may exhibit a length measured along the longitudinal axis of the fluid collection assembly 100 that may be at least about 10% of the length of the fluid collection assembly 100, such as about 25% to about 50%, about 40% to about 60%, about 50% to about 75%, about 65% to about 85%, or about 75% to about 95% of the length of the fluid collection assembly 100.

The opening 104 in the fluid impermeable barrier 102 may exhibit a width measured transverse to the longitudinal axis of the fluid collection assembly 100 that may be, when the fluid collection assembly 100 and the bladder 114 are in the first state, at least about 10% of the circumference of the fluid collection assembly 100, such as about 25% to about 50%, about 40% to about 60%, about 50% to about 75%, about 65% to about 85%, or about 75% to about 100% of the circumference of the fluid collection assembly 100. The opening 104 may exhibit a width that is greater than 50% of the circumference of the fluid collection assembly 100 since the vacuum (e.g., suction) through the conduit 128 pulls the fluid through the porous material 110 and into the conduit 128. As shown in FIGS. 1B and 1C, switching the bladder 114 from the first state to the second state increases the width of the opening 104. The increased width of the opening 104 may allow the opening 104 to receive more bodily fluids than if the opening 104 did not exhibit the increased width.

In some examples, the opening 104 may be vertically oriented (e.g., having a major axis parallel to the longitudinal axis of the fluid collection assembly 100). In some examples (not shown), the opening 104 may be horizontally oriented (e.g., having a major axis perpendicular to the longitudinal axis of the fluid collection assembly 100). In an example, the fluid impermeable barrier 102 may be configured to be attached to the individual, such as adhesively attached (e.g., with a hydrogel adhesive) to the individual. According to an example, a suitable adhesive is a hydrogel layer.

As previously discussed, the fluid impermeable barrier 102 may define fluid outlet 108 configured to remove bodily fluids from the chamber 106. The fluid outlet 108 is distinct from the opening 104 and the valve 120. In some examples, the fluid outlet 108 is sized to receive the conduit 128. The conduit 128 may be disposed in the chamber 106 via the fluid outlet 108. The fluid outlet 108 may be sized and shaped to form an at least substantially fluid tight seal against the conduit 128 or the at least one tube substantially preventing the bodily fluids from escaping the chamber 106.

The fluid impermeable barrier 102 may include markings thereon, such as one or more markings to aid a user in aligning the fluid collection assembly 100 on the patient. For example, a line on the fluid impermeable barrier 102 (e.g., opposite the opening 104) may allow a healthcare professional to align the opening 104 over the urethral opening of the patient. In examples, the markings may include one or more of alignment guide or an orientation indicator, such as a stripe or hashes. Such markings may be positioned to align the fluid collection assembly 100 to one or more anatomical features such as a pubic bone, etc.

As previously discussed, the fluid collection assembly 100 includes porous material 110 disposed in the chamber 106. The porous material 110 may cover at least a portion (e.g., all) of the opening 104. The porous material 110 is exposed to the environment outside of the chamber 106 through the opening 104. The permeable properties referred to herein may be wicking, capillary action, absorption, diffusion, or other similar properties or processes, and are referred to herein as “permeable” and/or “porous.” The porous material 110 may also wick the bodily fluids generally towards an interior of the chamber 106, as discussed in more detail below. The porous material 110 may include one or more of a fluid permeable membrane 130 or a fluid permeable support 132.

In an embodiment, at least a portion of the porous material 110 may be a wicking material configured to wick and/or allow transport of the bodily fluids away from the opening 104, thereby preventing bodily fluids from escaping the chamber 106. The porous material 110 may not include absorption of the bodily fluids into the w porous material 110. Put another way, substantially no absorption of the bodily fluids into the porous material 110 may take place after the wicking material is exposed to the bodily fluids. While no absorption is desired, the term “substantially no absorption” may allow for nominal amounts of absorption of the bodily fluids into the wicking material (e.g., absorbency), such as about 30 wt % or less of the dry weight of the wicking material, about 20 wt % or less, 10 wt % or less, about 7 wt % or less, about 5 wt % or less, about 3 wt % or less, about 2 wt % or less, about 1 wt % or less, or about 0.5 wt % or less of the dry weight of the wicking material.

The fluid collection assembly 100 may include the fluid permeable membrane 130 disposed in the chamber 106. The fluid permeable membrane 130 may cover at least a portion (e.g., all) of the opening 104. The fluid permeable membrane 130 may be composed to pull/push the bodily fluids away from the opening 104, thereby promoting fluid flow into the chamber 106, prevent fluid remaining on the vulva of the patient, and preventing the bodily fluids from escaping the chamber 106.

The fluid permeable membrane 130 may include any material that may be permeable to the bodily fluids. For example, the fluid permeable membrane 130 may include fabric, such as a gauze (e.g., a silk, linen, or cotton gauze), another soft fabric, or another smooth fabric. Forming the fluid permeable membrane 130 from gauze, soft fabric, and/or smooth fabric may reduce chaffing caused by the fluid collection assembly 100 and makes wearing the fluid collection assembly more comfortable. In an embodiment, the fluid permeable membrane 130 is formed from a flexible material, such as gauze, since the shape and/or size of the fluid permeable membrane 130 may change when the fluid collection assembly 100 and the bladder 114 switch between states, as shown in FIGS. 1B and 1C. In an embodiment, the fluid permeable membrane 130 may define a plurality of perforations or may be continuous (e.g., does not define perforations). In an embodiment, the fluid permeable membrane 130 defines at least one hole that is configured to allow the valve 120 to extend through the fluid permeable membrane 130.

The fluid collection assembly 100 may include the fluid permeable support 132 disposed in the chamber 106. The fluid permeable support 132 is configured to support the fluid permeable membrane 130 and maintain the shape of the chamber 106 since the fluid impermeable barrier 102 and the fluid permeable membrane 130 may be formed from a relatively foldable, flimsy, or otherwise easily deformable material. For example, the fluid permeable support 132 may be positioned so the fluid permeable membrane 130 is disposed between the fluid permeable support 132 and the fluid impermeable barrier 102. The fluid permeable support 132 may support and maintain the position of the fluid permeable membrane 130 and the shape of the chamber 106. The fluid permeable support 132 may include any material that may be permeable to the bodily fluids, such as any of the fluid permeable membrane 130 materials disclosed above. For example, the fluid permeable membrane 130 material(s) may be utilized in a more dense or rigid form than in the fluid permeable membrane 130 when used as the fluid permeable support 132. The fluid permeable support 132 may be formed from any fluid porous material that is less deformable than the fluid permeable membrane 130. For example, the fluid permeable support 132 may include a porous polymer (e.g., nylon, polyester, polyurethane, polyethylene, polypropylene, etc.) structure (e.g., spun fibers such as spun nylon fibers), nonwoven material, or a foam (e.g., an open cell foam). In some examples, the fluid permeable support 132 may be formed from a natural material, such as cotton, wool, silk, or combinations thereof. In such examples, the material may have a coating to prevent or limit absorption of the bodily fluids into the material, such as a water repellent coating. In some examples, the fluid permeable support 132 may be formed from fabric, felt, gauze, or combinations thereof.

In an embodiment, as illustrated, the bladder 114 is positioned between the fluid permeable membrane 130 and the fluid permeable support 132 (e.g., the bladder 114 is positioned in the chamber 106). The bladder 114 may be positioned between the fluid permeable membrane 130 and the fluid permeable support 132 since that at least some materials that form the fluid impermeable barrier 102 and the fluid permeable membrane 130 exhibit a flexibility that allows the fluid impermeable barrier 102 and the fluid permeable membrane 130 to accommodate the size and/or shape changes discussed above. The fluid permeable membrane 130 also covers the bladder 114 thereby protecting the bladder 114 from objects that may puncture the bladder 114. The fluid impermeable barrier 102 may also cover at least a portion of the bladder 114 thereby also protecting the bladder 114 from objects. The bladder 114 may be positioned in the chamber 106 so the bladder 114 does not obstruct or only slight obstructs bodily fluids from flowing through the fluid permeable membrane 130 to the fluid permeable support 132. For example, the bladder 114 may be positioned so, at most, only a small portion of the bladder 114 (e.g., at most 20%, at most 10%, or at most 5%) is not positioned between the fluid impermeable barrier 102 and the support 132.

In some examples, the fluid permeable membrane 130 may be optional. For example, the porous material 110 may include only the fluid permeable support 132. In such examples, the bladder 114 may be positioned within the fluid permeable support 132 since, for instance, at least some materials of the support 132 disclosed herein are flexible enough to accommodate the shape and/or size changes discussed herein. In some examples, the fluid permeable support 132 may be optionally omitted from the fluid collection assembly 100 and the porous material 110 may only include the fluid permeable membrane 130. In such examples, the bladder 114 may be positioned within the fluid permeable membrane 130.

In an embodiment, the fluid permeable membrane 130 and/or the fluid permeable support 132 are wicking materials. In such an embodiment, the fluid permeable support 132 may have a greater ability to wick the bodily fluids than the fluid permeable membrane 130, such as to move the bodily fluids inwardly from the fluid permeable membrane 130. In some examples, the wicking ability of the fluid permeable support 132 and the fluid permeable membrane 130 may be substantially the same. In an embodiment, the fluid permeable membrane 130 and/or the fluid permeable support 132 are non-wicking materials (e.g., absorbent and/or adsorbent materials).

In an embodiment, not shown, the fluid permeable membrane 130 and the fluid permeable support 132 may at least substantially completely fill the portions of the chamber 106 not occupied by the inflation device 112 and the conduit 128. In an embodiment, as shown in FIG. 1D, the fluid permeable membrane 130 and the fluid permeable support 132 may not substantially completely fill the portions of the chamber 106 not occupied by the inflation device 112 or the conduit 128. In such an embodiment, the fluid collection assembly 100 includes the fluid reservoir 133 disposed in the chamber 106.

The fluid reservoir 133 is a substantially unoccupied portion of the chamber 106. The fluid reservoir 133 may be defined between the fluid impermeable barrier 102 and at least one of the inflation device 112, the fluid permeable membrane 130, or the fluid permeable support 132. The bodily fluids in the chamber 106 may flow through the fluid permeable membrane 130 and/or fluid permeable support 132 to the fluid reservoir 133. The fluid reservoir 133 may retain of the bodily fluids. The bodily fluids in the chamber 106 may flow through the fluid permeable membrane 130 and/or fluid permeable support 132 and, optionally, to the fluid reservoir 133. The fluid impermeable barrier 102 may retain the bodily fluids in the fluid reservoir 133. The fluid reservoir 133 may be in a portion of the chamber 106 designed to be in a gravimetrically low point of the fluid collection assembly 100 when the fluid collection assembly 100 is worn.

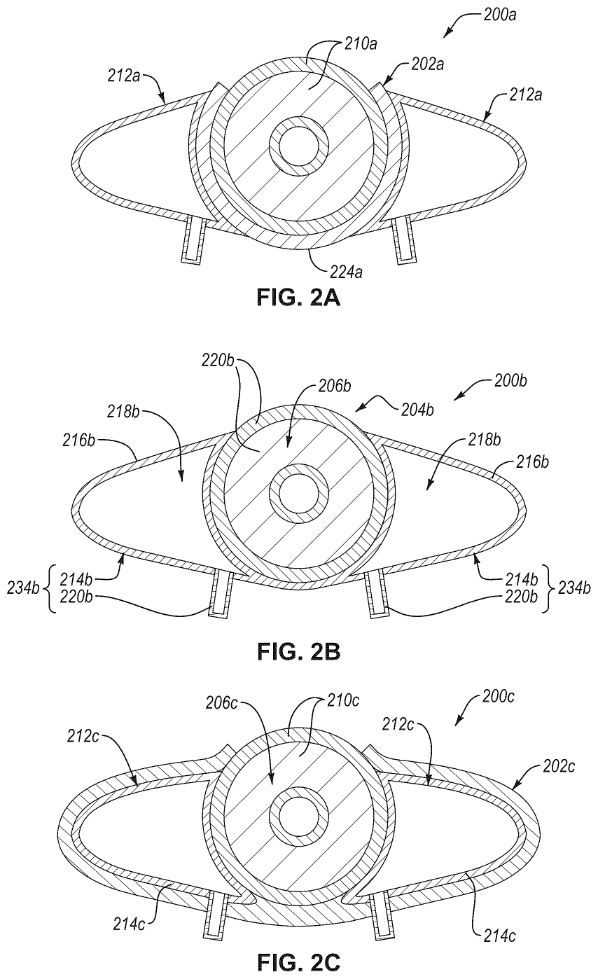

The inflation devices disclosed herein may be positioned in locations of the fluid collection assemblies disclosed herein other than between the fluid permeable membrane and the fluid permeable support. For example, FIGS. 2A to 2C are schematic cross-sectional views of different fluid collection assemblies that each have the inflation devices positioned differently, according to different embodiments. Except as otherwise disclosed herein, the fluid collection assemblies illustrated in FIGS. 2A-2C may be the same or substantially similar to any of the fluid collection assemblies disclosed herein. For example, the fluid collection assemblies illustrated in FIGS. 2A-2C may include a fluid impermeable barrier defining an opening, chamber, and fluid outlet; at least one porous material; and a conduit.

Referring to FIG. 2A, a fluid collection assembly 200a includes a fluid impermeable barrier 202a and at least one porous material 210a. The fluid impermeable barrier 202a includes an outer surface 224a. The fluid collection assembly 200a includes an inflation device 212a that is distinct from the fluid impermeable barrier 202a. The inflation device 212a is positioned on and attached to at least a portion of the outer surface 224a. For example, the inflation device 212a may be attached to the outer surface 224a using an adhesive, ultrasonic welding, heat staking, or any other suitable technique. Since the inflation device 212a is disposed on the outer surface 224a, switching the fluid collection assembly 202a and the inflation device 212a between different states will have limited effect on the size and shape of the fluid impermeable barrier 202a and the porous material 210a compared to the fluid impermeable barrier 102 and the fluid permeable membrane 130 shown in FIGS. 1A-1D. The fluid impermeable barrier 202a and the porous material 210a may, optionally, be formed from more rigid materials than the fluid impermeable barrier 102 and the fluid permeable membrane 130 shown in FIGS. 1A-1D. Positioning the inflation device 212a on the outer surface 224a may make manufacturing of the fluid collection assembly 200a easier since the inflation device 212a need not be positioned between two components of the fluid collection assembly 200a.

Referring to FIG. 2B, the fluid collection assembly 200b includes an inflation barrier 234b. The inflation barrier 234b is a fluid impermeable barrier and an inflation device integrally formed together. The inflation barrier 234b may exhibit any of the properties and functions as any of the fluid impermeable barriers and inflation devices disclosed herein. For example, the inflation barrier 234b may define an opening 204b, a chamber 206b, and a fluid outlet (not shown) and may be configured to prevent bodily fluids from leaking from the chamber 206b. The inflation barrier 234b may also include a bladder 214b and a valve 220b. The bladder 214b includes one or more walls 216b defining an interior region 218b. The inflation barrier 234b is configured to switch between one or more states (e.g., a first state and a second state) by adding or removing fluids from an inflation region 218 using the valve 220.

Switching the fluid collection assembly 202b and the inflation barrier 234b between different states will have limited effect on the size and shape of the porous material 210b compared to the fluid impermeable barrier 130 shown in FIGS. 1A-1C. The porous material 210b may, optionally, be formed from more rigid materials than the fluid permeable membrane 130 shown in FIGS. 1A-1C. Further, the inflation barrier 234b may make manufacturing of the fluid collection assembly 200b easier since the inflation barrier 234b need not be positioned between two components of the fluid collection assembly 200b and decreases the number of components that form the fluid collection assembly 200b.

Referring to FIG. 2C, the fluid collection assembly 200c includes a fluid impermeable barrier 202c and at least one porous material 210c. The fluid collection assembly 200c also includes an inflation device 212c that is distinct from the fluid impermeable barrier 202c. At least a portion of the inflation device 212c (e.g., at least the bladder 214c) is positioned between the fluid impermeable barrier 202c and the porous material 210c. The fluid impermeable barrier 202c may protect the inflation device 212c from objects that may puncture the inflation device 212c. Since the inflation device 212c is disposed between the fluid impermeable barrier 202c and the porous material 210c, switching the fluid collection assembly 202c and the inflation device 212c between different states will have limited effect on the size and shape of the porous material 210c compared to the porous material 110 shown in FIGS. 1A-1D. The porous material 210c may, optionally, be formed from more rigid materials than the fluid permeable membrane 130 shown in FIGS. 1A-1D. Positioning the inflation device 212c between the fluid impermeable barrier 202c and the porous material 210c may make manufacturing of the fluid collection assembly 200c easier since the inflation device 212c may be positioned in the chamber 206c before or while the porous material 210c is positioned in the chamber 206c. The method for forming the fluid collection assembly 200c does not require the additional step of positioning the inflation device 212c in the porous material 210c.

It is noted that the inflation devices disclosed herein may have positions other that the positions illustrated in FIGS. 1B and 2A-2C. For example, the inflation devices may be positioned within the fluid permeable membrane, within the fluid permeable support, between the fluid permeable support and the conduit, within the conduit, or integrally formed with the conduit.

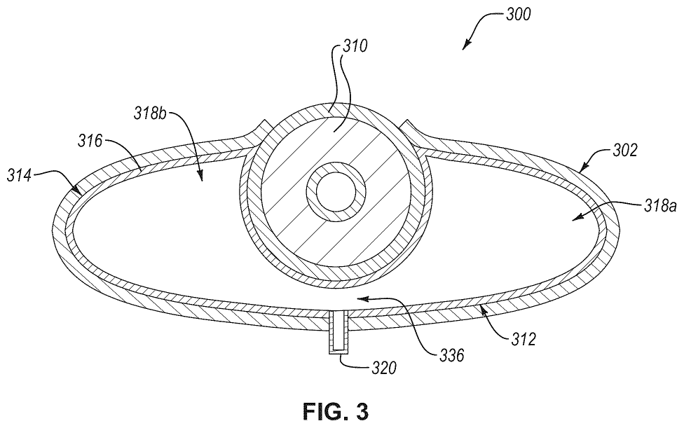

FIG. 3 is a cross-sectional schematic view of a fluid collection assembly 300 when the at least one bladder thereof is in the second state, according to an embodiment. Except as otherwise disclosed herein, the fluid collection assembly 300 is the same or substantially similar to any of the fluid collection assemblies disclosed herein. For example, the fluid collection assembly 300 includes a fluid impermeable barrier 302 and at least one porous material 310. The fluid collection assembly 300 also includes at least one inflation device 312. In an embodiment, as illustrated, at least a portion of the inflation device 312 is positioned between the fluid impermeable barrier 302 and the porous material 310. However, the inflation device 312 may include any of the other positions disclosed herein.

The inflation device 312 includes a bladder 314. The first bladder 314 includes one or more walls 316. The one or more walls 316 define a first inflation region 318a and a second inflation region 318b. The inflation device 312 includes at least one passageway 336 extending between and fluidly coupling the first inflation region 318a to the second inflation region 318b. For example, the one or more walls 316 defines the passageway 336. Inflation fluids present in the first inflation region 318a may flow to the second inflation region 318b by entering the passageway 336, flowing through the passage way 336, and then exiting the passageway 336 and vice versa. The pressure inside each of the first inflation region 318a and the second inflation region 318b remains the same thereby preventing over inflation or under inflation of one of the first inflation region 318a or the second inflation region 318b.

In an embodiment, the inflation device 312 may include a single valve 320 since the first inflation region 318a is in fluid communication with the second inflation region 318b. The valve 320 may be in direct fluid communication with one of the first inflation region 318a, the second inflation region 318b, or the passageway 336. However, it is noted that the inflation device 312 may include at least one valve 320 that is in direct fluid communication with at least two of the first inflation region 318a, the second inflation region 318b, or the passageway 336.

In an embodiment, inflation device 310 may include one or more additional inflation regions (not shown) that are in fluid communication with the first inflation region 318a and the second inflation region 318b through the passageway 336 or through one or more additional passageways. In an embodiment, the passageway 336 may include a passageway valve that selective permits or restricts fluid flow between the first inflation region 318a to the second inflation region 318b. For example, the passageway valve may be open (e.g., permit fluid flow between the first inflation region 318a and the second inflation region 318b) when it is desirable for the first inflation region 318a and the second inflation region 318b to exhibit the same pressure. However, the passageway valve may be closed when it is desirable to independently inflate at least one of the first inflation region 318a or the second inflation region 318b (e.g., when it is desirable for one of the first inflation region 318a or the second inflation region 318b to be inflated more than the other). The passageway valve may include any suitable valve, such as a mechanical valve or a clamp.

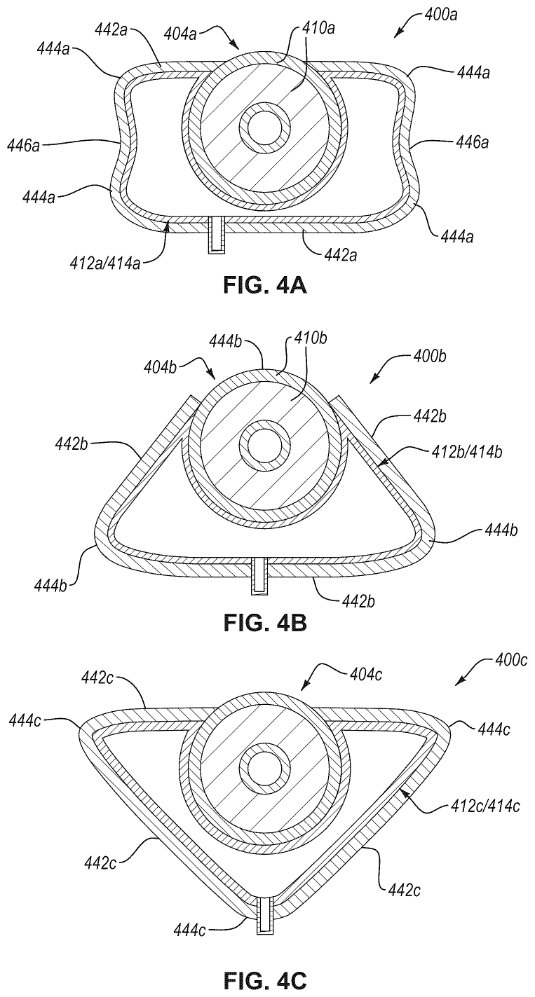

The fluid collection assemblies illustrated in FIGS. 1C and 2A-3 exhibit the same general oblong cross-sectional shape when the bladders of the fluid collection assemblies are in the second state. However, it is noted that the fluid collection assemblies may exhibit other cross-sectional shapes, without limitation. FIGS. 4A to 4C are schematic cross-sectional views of fluid collection assemblies exhibiting different shapes when the bladders of the fluid collection assemblies are in the second state, according to different embodiments. Except as otherwise disclosed herein, the fluid collection assemblies illustrated in FIGS. 4A-4C are the same or substantially similar to any of the fluid collection assemblies disclosed herein. For example, the fluid collection assemblies include fluid impermeable barrier, at least one porous material, and at least one inflation device. In an embodiment, as illustrated, the inflation device is positioned between the fluid impermeable barrier and the porous material. However, it is noted that the inflation device may have any of the positions disclosed herein.

Regarding FIG. 4A, a fluid collection assembly 400a includes an inflation device 412a. The inflation device 412a includes at least one bladder 414a. When the bladder 414a is in the second state, the fluid collection assembly 400a exhibits a generally hour-glass cross-sectional shape. Similarly, when the bladder 414a is in the second state, the bladder 414a may form a generally hour-glass cross-sectional shape that corresponds to the generally hour-glass cross-sectional shape of the fluid collection assembly 400a except that the bladder 414a defines a cutout in which some components of the fluid collection assembly 400a (e.g., the at least one porous material 410a) may be positioned. In an embodiment, as illustrated, the bladder 414a includes a plurality of bladders each in fluid communication with each other. In an embodiment, the bladder 414a may include a plurality of bladders not in fluid communication with each other. In an embodiment, the bladder 414a may include a single bladder.

The generally hour-glass shape of the fluid collection assembly 400a includes two opposing edges 442a, four apexes 444a at the ends of the edges 442a, and two concave surfaces 446a extending between adjacent apexes 444a. The opening 404a is formed on one edge 442a. The two apexes 444a that are most proximate to the opening 404a are configured to fit at least partially in the fold formed between the inner thighs and the rest of the pubic region of the patient. The concave surfaces 446a are configured to press against and generally correspond to the convex shape inner thighs of the patient. The ability of the two apexes 444a to fit at least partially into the fold formed between the inner thighs and the rest of the pubic region and the concave surfaces 446a to correspond to the concave inner thighs help secure the fluid collection assembly 400a to the patient, maintain the opening 404a against the vulva of the patient, and make the fluid collection assembly 400a more comfortable compared to some other shapes.

Regarding FIG. 4B, a fluid collection assembly 400b includes an inflation device 412b. The inflation device 412b includes at least one bladder 414b. When the bladder 414b is in the second state, the fluid collection assembly 400b exhibits a generally triangular cross-sectional shape. Similarly, when the bladder 414b is in the second state, the bladder 414b may also form a generally triangular cross-sectional shape that corresponds to the generally triangular cross-sectional shape of the fluid collection assembly 400b except that the bladder 414b defines a cutout in which some components of the fluid collection assembly 400b (e.g., the at least one porous material 410b) may be positioned. In an embodiment, as illustrated, the bladder 414b may include a single bladder. In an embodiment, the bladder 414b includes a plurality of bladders each in fluid communication with each other. In an embodiment, the bladder 414b may include a plurality of bladders not in fluid communication with each other.

The generally triangular cross-sectional shape of the fluid collection assembly 400b includes three edges 442b and three apexes 444b at the intersection of each edge 442b. The opening 404b is formed at one of the three apexes 444b which allows that porous material 410b to partially fit between the labia and to press more closely to the urethral opening of the patent. Positioning a portion of the porous material 410b between the labia helps secure the fluid collection assembly 400b to the patient.

FIG. 4C illustrates a fluid collection assembly 400c that, except as otherwise disclosed herein, in the same as the fluid collection assembly 400b of FIG. 4B. For example, when the bladder 414c of the inflation device 412c is in the second state, the fluid collection assembly 400c exhibits a generally triangular cross-sectional shape and the bladder 414c exhibits a corresponding generally triangular cross-sectional shape with a cutout. However, the opening 404c is formed in one edge 442c. Forming the opening 404c allows two of the apexes 444c that are closest to the opening 404c to fit into the fold between the inner thighs and the rest of the pubic region. Fitting the edges 442c into the fold helps secure the fluid collection assembly 400c to the patient. Further, the two edges 442c that do not include the opening 404c better conform to the convex shape of the inner thighs of the patient compared to the fluid collection assembly 400b of FIG. 4B which may make the fluid collection assembly 400c more comfortable to use.

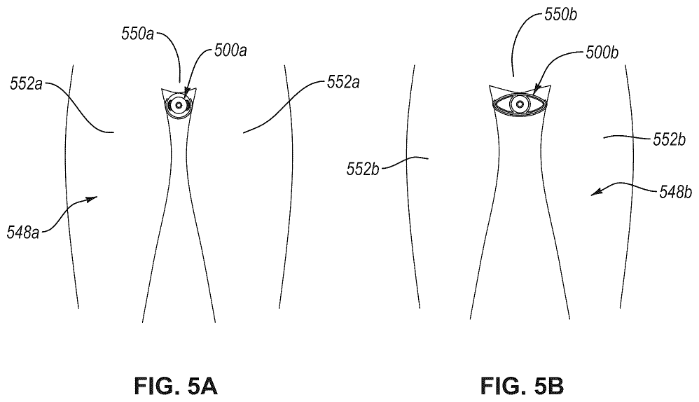

FIG. 5A is a schematic illustration of a first fluid collection assembly 500a being used with a first female patient 548a, according to an embodiment. During use, the first fluid collection assembly 500a is positioned between the thighs 552a of the first female patient 548a so the opening 504a of the first fluid collection assembly 500a is adjacent to the vulva 550a of the first female patient 548a. The first fluid collection assembly 500a may include any of the fluid collection assemblies disclosed herein that are configured to be used with a female patient. The thighs 552a of the first female patient 548a are sufficiently large that the first fluid collection assembly 500a presses against the thighs of the first female patient 548a even when the bladder of the first fluid collection assembly 500a is in the first state. The first fluid collection assembly 500a may be conformed to the vulva 550a and other portions of the pubic region of the first female patient 548a and the thighs 552a of the first female patient 548a may maintain the shape of the first fluid collection assembly 500a. The first fluid collection assembly 500a may be used with female patients whose thighs are sufficiently large that the first fluid collection assembly 500a need not be inflated even though the first fluid collection assembly 500a can be inflated.

FIG. 5B is a schematic illustrated of a second fluid collection assembly 500b being used with a second female patient 548b, according to an embodiment. During use, the second fluid collection assembly 500b is positioned between the thighs 552b of the second female patient 548b so the opening 504b of the second fluid collection assembly 500b is adjacent to the vulva 550b of the first female patient 548b. The second fluid collection assembly 500b is the same as the first fluid collection assembly 500a. The thighs 552b of the second female patient 548b are sufficiently small that the second fluid collection assembly 500b does not contact the thighs 552b or does not contact the thighs 552b with sufficient force to keep the second fluid collection assembly 500b conformed to the pubic region of the second female patient 548b. The bladder of the second fluid collection assembly 500b may be inflated from the first state to the second state. The second fluid collection assembly 500b is sufficiently inflated in the second state that the second fluid collection assembly 500b presses against the thighs 552b of the second female patient 548b with sufficient force that the thighs 552b keep the second fluid collection assembly 500b conformed to the pubic region. FIG. 5B illustrates how the fluid collection assemblies disclosed herein may be used with female patients that are underweight or have thin thighs.

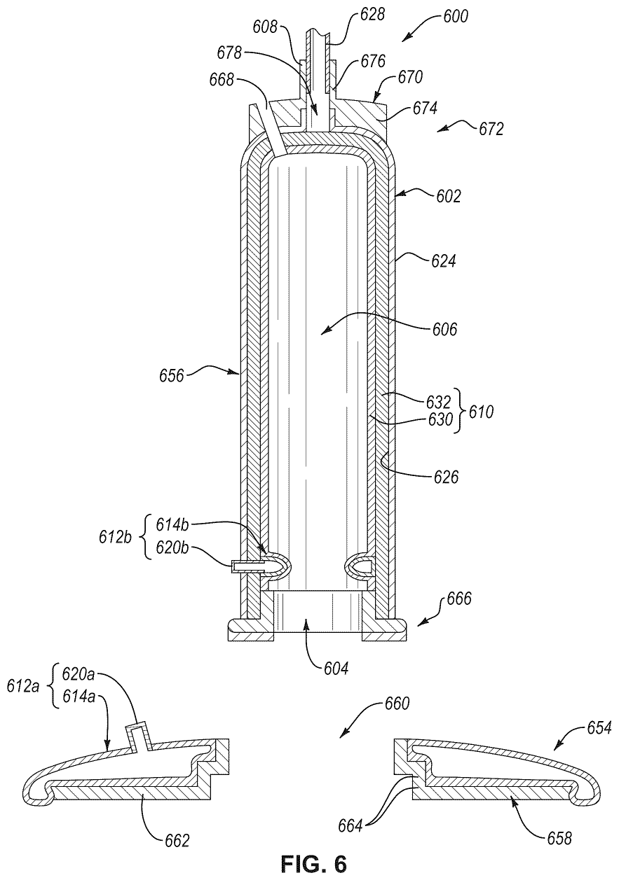

The fluid collection assemblies shown in FIGS. 1A-5B are examples of female fluid collection assemblies configured to collect bodily fluids from females (e.g., collection urine from a female urethral opening). However, the fluid collection assemblies, systems, and method disclosed herein may include male fluid collection assemblies shaped, sized, and otherwise configured to collection bodily fluids from males (e.g., collection urine from a penis). FIG. 6 is a cross-sectional view of a male fluid collection assembly 600, according to an embodiment.

The fluid collection assembly 600 includes a receptacle 654 and a sheath 656. The receptacle 654 is sized, shaped, and made of a material to be coupled to skin that surrounds the penis (mons pubis, testicles, perineum, etc.) and have the penis positioned therethrough. For example, the receptacle 654 may include an annular base 658 that defines an opening 660 in the receptacle 654. The annular base 658 is sized and shaped to be positioned around the penis and have the penis positioned therethrough. The annular base 658 may also be sized, shaped, made of a material, or otherwise configured to be coupled (e.g., adhesively attached, such as with a hydrogel adhesive) to the skin around the penis. In an example, the annular base 658 may exhibit the general shape or contours of the skin surface that the annular base 658 is selected to be coupled with. The annular base 658 may be flexible thereby allowing the annular base 658 to conform to any shape of the skin surface. The annular base 658 may include a laterally extending flange 662. The receptacle 654 also defines a hollowed region configured to receive (e.g., seal against) the sheath 656. For example, the receptacle 654 may include a longitudinally extending flange 664 that extends upwardly from the annular base 658. The longitudinally extending flange 664 may be tall enough to prevent the sheath 656 from being accidentally removed from the receptacle 654 (e.g., at least 0.25 cm tall, 1 cm tall, at least 9 cm tall, or at least 5 cm tall). The receptacle 654 is at a proximal region 666 (regarding a patient) of the fluid collection assembly 600.

The sheath 656 includes (e.g., may be formed from) a fluid impermeable barrier 602 sized and shaped to fit into the hollowed region of the receptacle 654. For example, the sheath 656 may be generally tubular or cup-shaped, as shown. The generally tubular or cup-shaped fluid impermeable barrier 602 may at least partially define the outer surface 624 of the sheath 656. The fluid impermeable barrier 602 may be similar or identical to the fluid impermeable barrier 102 as disclosed herein, in one or more aspects. For example, the fluid impermeable barrier 602 may be constructed of any of the materials disclosed herein for the fluid impermeable barrier 102. The fluid impermeable barrier 602 at least partially defines the chamber 606. For example, the inner surface 626 of the fluid impermeable barrier 602 at least partially defines the perimeter of the chamber 606. The chamber 606 may be similar or identical to the chamber 106 in one or more aspects. For example, the chamber 606 may at least temporarily retain fluids. As shown, the fluid collection assembly 600 may include at least one porous material 610. The porous material 610 may be similar or identical to the porous material 110 in one or more aspects. For example, the porous material 610 may include one or more of a fluid permeable membrane 630 or a fluid permeable support 632. The fluid impermeable barrier 602 may also define an opening 604 extending through the fluid impermeable barrier 602 configured to have a penis positioned therethrough.

The sheath 656 and fluid impermeable barrier 602 may also include at least one aperture 668 (e.g., vacuum relief hole) that allows the chamber 606 to remain substantially at atmospheric pressure. The at least one aperture 668 may be at any point on the sheath 656, such as near or nearer the opening 660. In some examples (not shown), the aperture 668 may extend through the cap 670 or be disposed beneath the cap 670. In some examples, the fluid collection assembly 600 may not include the aperture 668, such as when a more complete seal as desired for the chamber 606.

The sheath 656 also includes at least a portion of the conduit 628, such as at least partially disposed in the chamber 606. For example, the conduit 628 may extend from the sheath 656 at the distal region 672 to a proximal region 666 at least proximate to the opening 660. The proximal region 666 may be disposed near or on the skin around the penis or pubic area therearound. When a patient lays on their back, bodily fluids may aggregate near the opening 660 against the skin of the patient. The bodily fluids may be removed from the chamber 606 via the conduit 628.

In some examples, the fluid impermeable barrier 602 may be constructed of a material and/or have a thickness that allows the sheath 656 to collapse when placed under vacuum, such as to remove air around a penis in the fluid collection assembly 600 during use. In such examples, the conduit 628 may extend only to or into the distal region 672 in the chamber 606 (e.g., not through to the area adjacent the opening). In such examples, bodily fluids may be collected and removed from the fluid collection assembly 600 at the end nearest the aperture 668. In such examples, the at least one aperture may be located nearest the opening 660.

In an example, portions of the chamber 606 may be substantially empty due to the varying sizes and rigidity of the male penis. However, in some examples, the outermost regions of the chamber 606 (e.g., periphery of the interior regions of the sheath 656) may include porous material 610 (e.g., one or more of the fluid permeable membrane 630 and fluid permeable support 632). For example, the porous material 610 may be bonded to the inner surface 626 of the fluid impermeable barrier 602. The porous material 610 may be positioned (e.g., at the distal end of the chamber 606) to blunt a stream of urine from the penis thereby limiting splashing and/or to direct the fluid(s) to a selected region of the chamber 606. Since the chamber 606 is substantially empty (e.g., substantially the chamber 606 forms a reservoir), the fluids are likely to pool at a gravimetrically low point of the chamber 606. The gravimetrically low point of the chamber 606 may be at an intersection of the skin of an patient and the fluid collection assembly 600, a corner formed in the sheath 656, or another suitable location depending on the orientation of the patient.

The porous material 610 may include one or more of the fluid permeable membrane 630 or the fluid permeable support 632. The fluid permeable membrane 630 and the fluid permeable support 632 may be similar or identical to the fluid permeable membrane 130 or the fluid permeable support 132 as respectively disclosed herein, in one or more aspects such as material make-up or wicking ability. One or more of the fluid permeable membrane 630 or the fluid permeable support 632 may be disposed between the fluid impermeable barrier 602 and a penis inserted into the chamber 606. The fluid permeable membrane 630 may be positioned between the fluid impermeable barrier 602 and a penis inserted into the chamber 606, such as between the fluid permeable support 632 and penis of a wearer as shown. The fluid permeable support 632 may be positioned between the fluid permeable membrane 630 and the fluid impermeable barrier 602. The inner surface 626, optionally including the end of the chamber 606 substantially opposite the opening 604, may be covered with one or both the fluid permeable membrane 630 or the fluid permeable support 632. The fluid permeable support 632 or the fluid permeable membrane 630 may be affixed (e.g., adhered) to the fluid impermeable barrier 602. The fluid permeable support 632 or the fluid permeable membrane 630 may be affixed to each other. In some examples, the porous material 610 only includes the fluid permeable membrane 630 or the fluid permeable support 632.

The fluid collection assembly 600 includes one or more inflation devices in one or more portions thereof. For example, the fluid collection assembly 600 will be discusses as having a first inflation device 612a and a second inflation device 612b. However, it is noted that the fluid collection assembly 600 may only include a single inflation device (e.g., the first inflation device 612a, the second inflation device 612b, or another inflation device) or three or more inflation devices (e.g., the first inflation device 612a, a second inflation device 612b, and another inflation device). The inflation devices of the fluid collection assembly 600 may be similar or identical to any of the inflation devices disclosed herein in one or more aspects. For example, the first inflation device 612a may include at least one first bladder 614a and at least one first valve 620a and the second inflation device 612b may include at least one second bladder 614b and at least one second valve 620b.

The first inflation device 612a may be disposed on (as shown) or in the receptacle 654. In an embodiment, the first inflation device 612a increases the width (measure perpendicular to the longitudinal axis of the fluid collection assembly 600) of the receptacle when the pubic region of the patient is large enough to accommodate the increased width. The increase width of the receptacle 654 increases the surface area of the receptacle 654 that contacts the patient which may facilitate securement of the receptacle 654. For example, the increased surface area may allow for additional adhesive to be applied to the surface of the receptacle 654 that contacts the patient. In an embodiment, the first inflation device 612a changes the cross-sectional shape (measured perpendicular to the longitudinal axis of the fluid collection assembly 600) of the receptacle 654 which allows the receptacle 654 to conform to differently shaped pubic regions. In an embodiment, the first inflation device 612a increases a thickness of the receptacle 654 which may increase a contact pressure that, at least partially, secures the receptacle 654 to the pubic region.

The second inflation device 612b may be disposed on or in (as shown) the sheath 656. The second inflation device 612b may be configured to decrease a width of the chamber 606. For example, decreasing the width of the chamber 606 may cause the sheath 656 to contact a smaller diameter penis, a non-erect penis, or to conform to a penis that is transitioning between erect and non-erect states. Decreasing the width of the chamber 606 may inhibit bodily fluids from flowing away from the fluid outlet 608 thereby facilitating removal of the bodily fluids from the chamber 606.

In some examples, the fluid collection assembly 600 includes a cap 670 at a distal region 672. The cap 670 defines an fluid outlet 608 through which the fluids may be removed from the fluid collection assembly 600. The fluid outlet 608 is in fluid communication with the chamber 606. The cap 670 may be disposed over at least a portion of the distal region 672 of one or more of the fluid impermeable barrier 602 or the porous material 610. The cap 670 may be made of a polymer, rubber, or any other fluid impermeable material. The cap 670 may be attached to one or more of the fluid impermeable barrier 602, the porous material 610, or the conduit 628. The cap 670 may have a laterally extending flange 674 and a longitudinally extending flange 676. The laterally extending flange 674 may cover at least a portion of the distal region 672 of the fluid collection assembly 600. The longitudinally extending flange 676 may laterally extend a distance from the sheath 656. The longitudinally extending flange 676 is sized and configured to receive and fluidly seal against the conduit 628, such as within the fluid outlet 608. The conduit 628 may extend a distance within or through the cap 670, such as to the porous material 610, through the porous material 610, or to a point set-off from the porous material 610. In an example, the cap 670 may define a reservoir 678.

The reservoir 678 is an unoccupied portion of device such as in the cap 670 and is void of other material. In some examples, the reservoir 678 is defined at least partially by the porous material 610 and the cap 670. During use, the fluids in the chamber 606 may flow through the porous material 610 to the reservoir 678. The reservoir 678 may store at least some fluids and/or position the fluids for removal by the conduit 628. In some examples, at least a portion of the porous material 610 may extend continuously between at least a portion of the opening of the fluid outlet 608 and chamber 606 to wick any fluid from the opening directly to the reservoir 678.

In some examples (not shown), the fluid impermeable barrier 602 may be disposed on or over the cap 670, such as enclosing the cap 670 within the chamber 606.

In some examples, the sheath 656 may include at least a portion of the conduit 628, such as at least partially disposed in the chamber 606. For example, the conduit 628 may extend from the sheath 656 to a region at least proximate to the opening 660. The inlet of the conduit 628 may be positioned adjacent to the annular base 658. The inlet of the conduit 628 may be positioned to be adjacent or proximate to the gravimetrically low point of the chamber 606, such as adjacent to the annular base 658. For example, the inlet may be co-extensive with or offset from the opening 660. In examples, the inlet may be positioned adjacent to the distal region 672 of the sheath 656 (e.g., substantially opposite the opening).

The proximal region 666 may be disposed near or on the skin around the penis and the inlet of the conduit 628 may be positioned in the proximal region 666. The outlet of the conduit 628 may be directly or indirectly coupled to a vacuum source. Fluid may be removed from the proximal region 666 of the chamber 606 via the conduit 628.

The receptacle 654, the sheath 656, the cap 670, and the conduit 628 may be attached together using any suitable method. For example, at least two of the receptacle 654, the sheath 656, the cap 670, or the conduit 628 may be attached using at least one of an interference fit, an adhesive, stitching, welding (e.g., ultrasonic welding), tape, any other suitable method, or combinations thereof.

In some examples (not shown), the fluid collection assembly 600 may have a one piece design, with one or more of the sheath 656, the receptacle 654, and the cap 670 being a single, integrally formed piece.

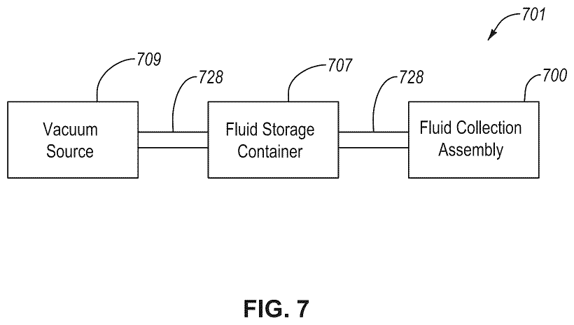

Also as shown, the conduit 628 may be at least partially disposed with the chamber of a fluid collection assembly. The conduit 628 may extend from the distal region 672 to the proximal region 666. For example, the conduit 628 may extend through the cap 670 to a point adjacent to the receptacle 654. The conduit 628 is sized and positioned to be coupled to a fluid storage container or the vacuum source (FIG. 7). An outlet of the conduit 628 may be operably coupled to the vacuum source, directly or indirectly. The inlet of the conduit 628 may be positioned within the chamber 606 such as at a location expected to be at the gravimetrically low point of the fluid collection assembly during use. By positioning the inlet in a location expected to be at the gravimetrically low point of the fluid collection assembly when worn by the user, fluids introduced into the chamber 606 may be removed via the conduit 628 to prevent pooling or stagnation of the fluid within the chamber 606.

In some examples, the vacuum source may be remotely located from the fluid collection assembly 600. In such examples, the conduit 628 may be fluidly connected to the fluid storage container, which may be disposed between the vacuum source and the fluid collection assembly 600.

During operation, a male using the fluid collection assembly 600 may discharge fluid(s) (e.g., urine) into the chamber 606. The fluid(s) may pool or otherwise be collected in the chamber 606. At least some of the fluid(s) may be pulled through the interior of the conduit 628 via the inlet. The fluid may be drawn out of the fluid collection assembly 600 via the vacuum/suction provided by the vacuum source. During operation, the aperture 668 may substantially maintain the pressure in the chamber 606 at atmospheric pressure even though fluid is introduced into and removed from the chamber 606.

Additional examples of male fluid collection assemblies that may include one or more inflation devices are disclosed in U.S. patent application Ser. No. 16/433,773 filed on Jun. 6, 2019, the disclose of which is incorporated herein, in its entirety, by this reference.

FIG. 7 is a block diagram of a system 701 for fluid collection, according to an embodiment. The system 701 includes a fluid collection assembly 700, a fluid storage container 707, and a vacuum source 709. The fluid collection assembly 700, the fluid storage container 707, and the vacuum source 709 may be fluidly coupled to each other via one or more conduits 728. For example, fluid collection assembly 700 may be operably coupled to one or more of the fluid storage container 707 or the vacuum source 709 via the conduit 728. Fluid (e.g., urine or other bodily fluids) collected in the fluid collection assembly 700 may be removed from the fluid collection assembly 700 via the conduit 728 which protrudes into the fluid collection assembly 700. For example, an inlet of the conduit 728 may extend into the fluid collection assembly 700, such as to a reservoir. The outlet of the conduit 728 may extend into the fluid collection assembly 700 or the vacuum source 709. Suction force may be introduced into the chamber of the fluid collection assembly 700 via the inlet of the conduit 728 responsive to suction (e.g., vacuum) force applied at the outlet of the conduit 728.

The suction force may be applied to the outlet of the conduit 728 by the vacuum source 709 either directly or indirectly. The suction force may be applied indirectly via the fluid storage container 707. For example, the outlet of the conduit 728 may be disposed within the fluid storage container 707 and an additional conduit 728 may extend from the fluid storage container 707 to the vacuum source 709. The vacuum source 709 may apply suction to the fluid collection assembly 700 via the fluid storage container 707. The suction force may be applied directly via the vacuum source 709. For example, the outlet of the conduit 728 may be disposed within the vacuum source 709. An additional conduit 728 may extend from the vacuum source 709 to a point outside of the fluid collection assembly 700, such as to the fluid storage container 707. In such examples, the vacuum source 709 may be disposed between the fluid collection assembly 700 and the fluid storage container 707.

The fluid collection assembly 700 may be similar or identical to any of the fluid collection assemblies disclosed herein in one or more aspects. The fluid collection assembly 700 may be shaped and sized to be positioned adjacent to a female urethral opening or receive a penis.

The fluid storage container 707 is sized and shaped to retain a fluid. The fluid storage container 707 may include a bag (e.g., drainage bag), a bottle or cup (e.g., collection jar), or any other enclosed container for storing bodily fluid(s) such as urine. In some examples, the conduit 728 may extend from the fluid collection assembly 700 and attach to the fluid storage container 707 at a first point. An additional conduit 728 may attach to the fluid storage container 707 at a second point thereon and may extend and attach to the vacuum source 709. A vacuum (e.g., suction) may be drawn through fluid collection assembly 700 via the fluid storage container 707. Fluid, such as urine, may be drained from the fluid collection assembly 700 using the vacuum source 709.

The vacuum source 709 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 709 may provide a vacuum or suction to remove fluid from the fluid collection assembly 700. In some examples, the vacuum source 709 may be powered by one or more of a power cord (e.g., connected to a power socket), one or more batteries, or even manual power (e.g., a hand operated vacuum pump). In some examples, the vacuum source 709 may be sized and shaped to fit outside of, on, or within the fluid collection assembly 700. For example, the vacuum source 709 may include one or more miniaturized pumps or one or more micro pumps. The vacuum sources 709 disclosed herein may include one or more of a switch, a button, a plug, a remote, or any other device suitable to activate the vacuum source 709.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. (canceled)

2. A fluid collection assembly, comprising:
a fluid impermeable barrier defining a chamber, at least one opening, and at least one fluid outlet;
at least one porous material disposed in the chamber;
at least one inflation device including at least one bladder and at least one valve, the at least one bladder including one or more walls defining at least one interior region, the at least one valve configured to allow at least one inflation fluid to flow at least into the at least one interior region to switch the at least one bladder from a first state to at least a second state, wherein an amount of the at least one inflation fluid present in the at least one interior region is greater when the at least one bladder is in the second state than when the at least one bladder is in the first state, wherein switching the at least one bladder between the first state and the second state changes least one of a width or a cross-sectional shape of the fluid collection assembly, wherein the width and the cross-sectional shape are measured perpendicularly to the longitudinal axis of the fluid collection assembly; and
a conduit disposed in the fluid outlet.

3. The fluid collection assembly of claim 2, wherein at least partially of the at least one inflation device is attached to at least a portion of at least one outer surface of the fluid impermeable barrier.

4. The fluid collection assembly of claim 2, wherein at least a portion of the at least one inflation device is positioned between the fluid impermeable barrier and the at least one porous material.

5. The fluid collection assembly of claim 2, wherein:
the at least one porous material includes at least one fluid permeable membrane and at least one fluid permeable support; and
at least a portion of the inflation device is positioned between the at least one preamble membrane and the at least one fluid permeable support.

6. The fluid collection assembly of claim 2, wherein the at least one bladder includes a plurality of bladders and the at least one inflation device includes at least one passageway extending between at least two of the plurality of inflation devices.

7. The fluid collection assembly of claim 2, wherein the at least one inflation device includes two inflation devices, the conduit positioned between the two inflation devices.

8. The fluid collection assembly of claim 7, wherein the fluid impermeable barrier includes a back wall opposite the at least one opening and two lateral walls extending from the at least one opening to the back wall, each the two inflation devices attached to, abutting, or integrally formed with one of the two lateral walls.

9. The fluid collection assembly of claim 2, wherein the fluid impermeable barrier includes a back wall opposite the at least one opening, the at least one valve extending through, directly from, or adjacent to the back wall.

10. The fluid collection assembly of claim 2, wherein the fluid impermeable barrier includes a back surface opposite the at least one opening, the at least one valve extending from or near the back surface.

11. The fluid collection assembly of claim 2, wherein the at least one valve includes a check valve.

12. The fluid collection assembly of claim 2, wherein the at least one valve includes a two-way valve.

13. The fluid collection assembly of claim 2, wherein the fluid collection assembly exhibits a first width when the at least one inflation element is in the first state and a second width when the at least one bladder is in the second state, wherein the first width is less than the second width, the first width and the second width measured perpendicular to a longitudinal axis of the fluid collection assembly.

14. The fluid collection assembly of claim 2, wherein the fluid collection assembly exhibits a first cross-sectional shape when the at least one inflation element is in the first state and a second cross-sectional shape when the at least one bladder is in the second state, wherein the first shape is different than the second shape, the first cross-sectional shape and the second cross-sectional shape measured perpendicular to the longitudinal axis of the fluid collection assembly.

15. The fluid collection assembly of claim 14, wherein the first cross-sectional shape is generally circular and the second cross-sectional shape is one of generally oblong, generally triangular, or generally hourglass shaped.

16. The fluid collection assembly of claim 2, wherein the fluid collection assembly exhibits a generally triangular cross-sectional shape when in at least one of the first state or the second state, the generally triangular cross-sectional shape measured perpendicular to the longitudinal axis of the fluid collection assembly.

17. The fluid collection assembly of claim 16, wherein the at least one opening is at an edge of the generally triangular cross-sectional shape.

18. The fluid collection assembly of claim 16, wherein the at least one opening is at an apex of the generally triangular cross-sectional shape.

19. The fluid collection assembly of claim 2, wherein the conduit is separate from the at least one inflation device.

20. A fluid collection assembly, comprising:
a fluid impermeable barrier defining a chamber, at least one opening, and at least one fluid outlet;
at least one porous material disposed in the chamber;
at least one inflation device including at least one bladder and at least one valve, the at least one bladder including one or more walls defining at least one interior region, the at least one valve configured to selectively permit at least one inflation fluid to flow into and out of the at least one interior region to switch the at least one bladder between a first state and at least a second state, wherein an amount of the at least one inflation fluid present in the at least one interior region is greater when the at least one bladder is in the second state than when the at least one bladder is in the first state, and wherein the fluid collection assembly exhibits a first cross-sectional shape when the at least one inflation element is in the first state and a second cross-sectional shape when the at least one bladder is in the second state, the first cross-sectional shape and the second cross-sectional shape measured perpendicular to a longitudinal axis of the fluid collection assembly.

21. A fluid collection assembly, comprising:
a fluid impermeable barrier defining a chamber, at least one opening, and at least one fluid outlet;
at least one porous material disposed in the chamber;
at least one inflation device including at least one bladder and at least one valve, the at least one bladder including one or more walls defining at least one interior region, the at least one valve configured to selectively permit at least one inflation fluid to flow into and out of the at least one interior region to switch the at least one bladder between a first state and at least a second state, wherein an amount of the at least one inflation fluid present in the at least one interior region is greater when the at least one bladder is in the second state than when the at least one bladder is in the first state, and wherein the fluid collection assembly exhibits a first width when the at least one inflation element is in the first state and a second width when the at least one bladder is in the second state, wherein the first width is less than the second width, the first width and the second width measured perpendicular to a longitudinal axis of the fluid collection assembly.
